(19) United States
(12) Patent Application Publication
(10) Pub. No.: US 2009/0175926 A1
(43) Pub. Date: Jul. 9, 2009

(54) TOPICAL BURN TREATMENT

(76) Inventor: Theodore P. Adams, Edina, MN (US)

Correspondence Address: NIKOLAI & MERSEREAU, P.A., 900 SECOND AVENUE SOUTH, SUITE 820, MINNEAPOLIS, MN, 55402, US

(21) Appl. No.: 11/970,606

(22) Filed: Jan. 8, 2008

(51) Int. Cl. A61K 9/70; A61K 9/14; A61P 17/02

(52) U.S. Cl. 424/443; 424/499

(57) ABSTRACT

In some embodiments, a method of reducing the possibility of infection in a burn may include one or more of the following steps: (a) identifying a burn wound, (b) applying topically a molecular sieve agent to the burn wound, and (c) allowing the burn wound to scab over due to absorption of moisture.

### FIELD OF THE INVENTION

Embodiments of the present invention generally relate to medical treatments. Particularly, embodiments of the present invention relate to topical medical treatments. More particularly, embodiments of the present invention relate to topical treatment of burns.

### BACKGROUND

In medicine, a burn may be an injury caused by heat, cold, electricity, chemicals, friction, or radiation (e.g., sunburn). Burns usually limited to redness (erythema), a white plaque, and minor pain at the site of injury are first degree burns. These burns usually extend only into the epidermis. Burns filled with clear fluid, having superficial blistering of the skin, and involving more or less pain depending on the level of nerve involvement are second degree burns. Second-degree burns involve the superficial (papillary) dermis and may also involve the deep (reticular) dermis layer.

Burns having charring of the skin and producing hard leather-like eschars are third degree burns. An eschar is a scab separated from the unaffected part of the body. Frequently, there is also purple fluid and/or pus. These types of burns are often painless, because nerve endings have been destroyed in the burned areas. Hair follicles and sweat glands may also be lost and third degree burns often result in scarring. Burns in which much or all of the dermis is lost often exposing and burning the muscle and possibly bone underneath are fourth degree burns. These burns usually present irreversible damage to the skin and there is no sensation in the burn area as a result. These types of burns will definitely require hospitalization. While skin grafting is usually needed to close up the areas, amputation of the extremity may be required. Often, fourth degree burns can be fatal.

Burns having charring or burning away of the muscle are fifth degree burns. These types of burns may leave bone exposed; thoroughly roasting surviving muscle tissue to the point of immobility, and burning the bone. Burns in which all muscle tissue in the area are burned away leaving nothing but charred bone are sixth degree burns. In some instances the bone tissue may be partially or completely burned away as well though this would require extreme conditions. Sixth degree burns are the highest burn category.

When the first layer of skin has been burned through and the second layer of skin (dermis) also is burned, the injury is termed a second-degree burn. Blisters develop and the skin takes on an intensely reddened, splotchy appearance. Second-degree burns produce severe pain and swelling. If the second-degree burn is no larger than 2 to 3 inches in diameter, it can be treated as a minor burn. If the burned area is larger or if the burn is on the hands, feet, face, groin, or buttocks, or over a major joint, one should get medical help immediately.

For minor burns, including second-degree burns limited to an area no larger than 2 to 3 inches in diameter, a person can take the following traditional actions: Cool the burn. Hold the burned area under cold running water for at least five minutes, or until the pain subsides. If this is impractical, immerse the burn in cold water or cool it with cold compresses. Cooling the burn reduces swelling by conducting heat away from the skin.

The burn can be covered with a sterile gauze bandage. Fluffy cotton should not be used as it may irritate the skin. Wrap the gauze loosely to avoid putting pressure on burned skin. Bandaging keeps air off the burned skin, reduces pain, and protects blistered skin. The burn victim can take an over-the-counter pain reliever. These include aspirin, ibuprofen, (Advil®, Motrin®, others), naproxen (Aleve®) or acetaminophen (Tylenol®, others). A local anesthetic can be sufficient in managing pain of smaller first-degree and second-degree burn. Lidocaine can be administered to the spot of injury and will generally negate most pain.

While these traditional methods of treating a first or second degree burn have proven to be effective these treatments do have their shortcomings. First the traditional treatment can require the burn be covered with a dressing. Dressings are not always available and this adds an extra step to the treatment of the burn. Second, while traditional pain relievers have proven effective, it does take some time for the pain relievers to work. Thus the burn victim is in a significant amount of pain for some time. Further, the traditional methods of burn treatment require a significant amount of time to heal and there is the issue of scarring.

Therefore, it is desirable to provide a burn treatment method reducing the amount of materials needed by personal attending to the burn. It is also desirable to have a burn treatment method which significantly reduces the amount of pain as well as the time it takes for the pain to subside. It is also desirable to have a burn treatment method which allows the burn to heal significantly faster and reduce scarring.

### SUMMARY OF THE INVENTION

In some embodiments, a method for treating a burn may include one or more of the following steps: (a) identifying a burn wound, and (b) applying a molecular sieve agent to the burn wound.

In some embodiments, a method for reducing pain associated with a burn may include one or more of the following steps: (a) identifying a burn wound, and (b) applying a molecular sieve agent to the burn wound.

In some embodiments, a method of reducing the possibility of infection in a burn may include one or more of the following steps: (a) identifying a burn wound, (b) applying topically a molecular sieve agent to the burn wound, and (c) allowing the burn wound to scab over due to absorption of moisture.

In some embodiments, a method of treating burns may include one or more of the following steps: (a) identifying a burn wound, and (b) applying topically a molecular sieve agent to the burn wound.

### DESCRIPTION OF THE DRAWINGS

FIG. 1A shows a diagrammatic representation of a first degree burn;

FIG. 1B shows a diagrammatic representation of a second degree burn;

FIG. 1C shows a diagrammatic representation of a third degree burn;

FIGS. 2A-F show a diagrammatic representation of a burn wound being treated by a topical hemostatic agent in an embodiment of the present invention; and

FIG. 3 shows a flow diagram of a method of treating a burn wound in embodiments of the present invention.

### DESCRIPTION OF THE PREFERRED EMBODIMENT

The following discussion is presented to enable a person skilled in the art to make and use the present teachings. Various modifications to the illustrated embodiments will be readily apparent to those skilled in the art, and the generic principles herein may be applied to other embodiments and applications without departing from the present teachings. Thus, the present teachings are not intended to be limited to embodiments shown, but are to be accorded the widest scope consistent with the principles and features disclosed herein. The following detailed description is to be read with reference to the figures, in which like elements in different figures have like reference numerals. The figures, which are not necessarily to scale, depict selected embodiments and are not intended to limit the scope of the present teachings. Skilled artisans will recognize the examples provided herein have many useful alternatives and fall within the scope of the present teachings. While the description below is presented predominately to the treatment of first, second, and third degree burns, it is fully contemplated the medical treatment discussed herein could be utilized for most any burn, first through sixth, without departing from the spirit of the invention.

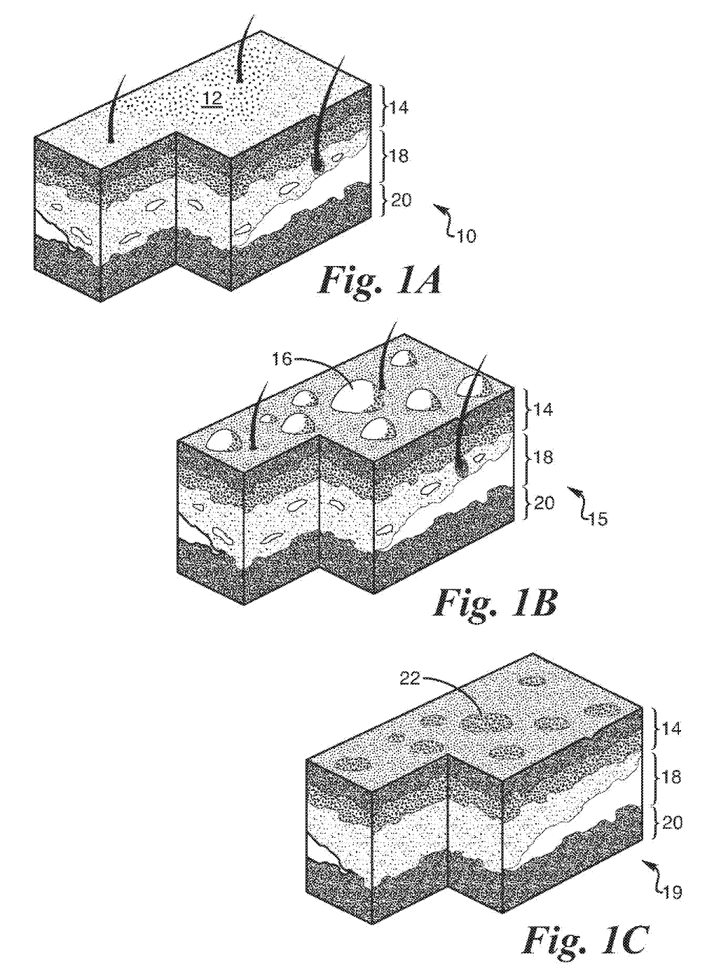

With reference to FIGS. 1A-C, diagrammatic representations of first, second, and third degree burns are shown. As discussed above, first degree burns 10 are usually limited to redness 12 (erythema), a white plaque, and minor pain at the site of injury. These burns usually extend only into epidermis 14. Second degree burns 15 additionally fill with clear fluid or pus (a generally viscous, yellowish-white fluid formed in infected tissue, consisting of white blood cells, cellular debris, and necrotic tissue), have superficial blistering 16 of the skin, and can involve more or less pain depending on the level of nerve involvement. Second-degree burns 15 involve the superficial (papillary) dermis 18 and may also involve the deep (reticular) dermis layer 20. Third degree burns 19 additionally have charring of the skin, and produce hard, leather-like eschars 22. Frequently, there is also pus.

The inventor has discovered using hemostatic agents, typically used to coagulate blood, for use as a treatment for certain burn wounds can reduce pain and increase healing of the burns. Some moderately severe burns (second degree) cause open festering wounds unable to readily scab over and they heal slowly. These burns are very painful. By using a molecular sieve type hemostatic agent, as will be discussed in more detail below, pain associated with the burn wound can be reduced. The burn scabs over very quickly thereby lessening the risk of infection. This helps the burn heal faster and also helps prevent scarring.

In one embodiment of the present invention, MPH® (Microporous Polysaccharide Hemispheres) is used to treat burn wounds. Other types of molecular sieves such as zeolite (a mineral used as a hemostatic agent) and powdered polyacrylamide (water crystals) which rapidly absorbs water perform the function similarly, but with undesirable side effects.

The molecular sieve, in powdered form, is spread over the burn area. By absorbing water from the open wound, and thereby causing other bio-matter in the pus to aggregate around the particles, it forms a scab quickly. Thus there is no need for the wound to be covered with a dressing. The sealing of the wound initiates almost immediate significant reduction in pain associated with the burn. Further, it also reduces the possibility of infections thereby enabling the burn to heal faster and with less scarring than if left unattended.

The molecular sieve can initiate almost instantaneous scabbing at burn sites, particularly in the presence of profuse seeping of burn wounds. The technology consists of an engineered biopolymeric, microporous particle, with a controlled pore size, which acts as a sieve to dehydrate the burn site and thus serve to accelerate the natural healing process. Using this technology, healing has been demonstrated to initiate within as little as a few seconds to one minute, compared to longer periods of time required by traditional methods. Applied topically, the material gels rapidly with no tissue irritation creating a protected environment for fluid coagulation and healing.

Molecular sieves may be used for the enhancement of the healing of burns in animals, including mammals, avians, and reptiles. They have a porous particulate material which is applied to the burn when there is moisture, such as pus (e.g., where it may wet the particles). The particles may be applied to the burn area either as a free flowing powder of the particles, a dry spray of particles, or aerosol of the particles as an association of particles in or on a carrier (e.g., a web, tape, fabric, foam, reticulated foam, or film), and may optionally contain conventional clotting agents with the particles. The particle application should enable direct contact of the particles with moisture. The use of the particles on the surface of a film with surface facing the burn would be acceptable. In orientation, the pus/powder combination would clot at the burn site.

An alternate embodiment has the particles located within a thin, light fibrous mass so as the particles enhanced clotting, the fibers would remain within the region of clotting, and strengthen the clot. The fibers could also be used to assist in carrying optional materials (e.g., antibiotics) to the burn site. One type of desirable materials of this last format would have a woven, non-woven or knitted fibrous sheet (e.g., less than 1 mm in thickness, e.g., 0.05 to 0.5 mm, or 0.1 to 0.5 mm thick) with the fabric having a porosity of at least 30% (e.g., 30-95%, 40-95%, or 50-95% porosity), with at least a portion of the porosity filled with the clot enhancing particles described for use in the practice of embodiments of the present invention. The particles may be carried within the structure of the fabric or bonded to the fibers, filaments, or yarns of the fibrous material (taking care not to completely fill the pores of the particles with any binder used).

The particles may generally have a size of from about 1 to 1000 micrometers, or 1 to 500 micrometers, but the size may be varied by one ordinarily skilled in the art to suit a particular use or type of patient and depending on the ability of a carrier to support the particles with their optional selection of sizes. Examples of specific materials useful in the practice of the present invention comprise porous materials from within the classes of polysaccharides, cellulosics, polymers (natural and synthetic), inorganic oxides, ceramics, zeolites, glasses, metals, and composites. Good materials are, of course, non-toxic and are provided as a sterile supply. The polysaccharides are desired because of their ready availability and modest cost. The porous particulate polysaccharides may be provided as starch, cellulose, and/or pectins, and even chitin may be used (animal sourced from shrimp, crab and lobster, for example). Glycosaccharides or glycoconjugates which are described as associations of the saccharides with either proteins (forming glycoproteins, especially glycolectins) or with a lipid (glycolipid) are also useful. These glycoconjugates appear as oligomeric glycoproteins in cellular membranes. In any event, all of the useful materials could be porous enough to allow blood or pus liquid and low molecular weight blood components to be adsorbed onto the surface and/or absorbed into the surface of the particles. Porosity through the entire particle is often more easily achieved rather than merely etching the surface or roughening the surface of the particles.

Ceramic materials may be provided from the sintering, or sol-gel condensation or dehydration of colloidal dispersions of inorganic oxides such as silica, titanium dioxide, zirconium oxide, zinc oxide, tin oxide, iron oxide, cesium oxide, aluminum oxide and oxides of other metal, alkaline earth, transition, or semimetallic chemical elements, and mixtures thereof. By selection of the initial dispersion size or sol size of the inorganic oxide particles, the rate of dehydration, the temperature at which the dehydration occurs, the shear rate within the composition, and the duration of the dehydration, the porosity of the particles and their size can be readily controlled.

With regard to cellulosic particles, the natural celluloses or synthetic celluloses (including cellulose acetate, cellulose butyrate, cellulose propionate, etc.) may be exploded or expanded according to techniques described in U.S. Pat. No. 5,817,381, herein incorporated by reference in its entirety, and other cellulose composition treating methods described therein which call provide porous particles, fibers and microfibers of cellulose based materials. Where the porous materials, whether of cellulose or other compositions, have a size which may be too large for a particular application, the particles may be ground or milled to an appropriate size. This can be done by direct mortar and pestle milling, ball milling, crushing (as long as the forces do not compress out all of the porosity), fluidized bed degradation and size reduction, and any other available physical process. Where the size of the raw material should be larger than the particle size provided, the smaller particles may be aggregated or bound together under controlled shear conditions with a binder or adhesive until the average particle size is within the desired range.

Porosity may be added to many materials by known manufacturing techniques, such as 1) co-dispersion with a differentially soluble material, and subsequent dissolution of the more soluble material, 2) particle formation from an emulsion or dispersion, with the liquid component being evaporated or otherwise removed from the solid particle after formation, 3) sintering of particles so as to leave porosity between the sintered or fused particles, 4) binding particles with a slowly soluble binder and partially removing a controlled amount of the binder, 5) providing particles with a two component, two phase system where one component is more readily removed than another solid component (as by thermal degradation, solubilization, decomposition, chemical reaction such as, chemical oxidation, aerial oxidation, chemical decomposition, etc.), and other known processes for generating porosity from different or specific types of compositions and materials. Where only surface porosity is needed in a particular clot promoting format, surface etching, or abrasion may be sufficient to provide the desired surface porosity.

A particularly desirable and commercially available material comprises polysaccharide beads, such as dextran beads which are available as Sephadex® beads from Pharmacia Labs and sold Debrisan. These are normally used in surgery as an aid to debridement of surfaces to help in the removal of damaged tissue and scar tissue from burns. The application of this type of porous bead (and the other types of porous beads to burns with blood or pus thereon) was intended as an absorbent has not been found not to be effective in promoting fluid coagulation, or speeding up the formation of scabs.

The porous particles or porous beads may be directly applied to burns or held in place by pressure. The beads or particles may be free flowing or be supported on or in a containment system. For example, the particles may be adhered to the surface of a sheet or film which is applied (e.g., contacted, wrapped, adhered, secured, affixed, or otherwise placed into a position where moisture on the burn area will be absorbed by the porous particles or porous beads) to areas of a burn with moisture. The particles may also be provided in a form where the porous particles or porous beads may be interspersed with fibers, filaments or other particles in a self-supporting structure, entangled within the fibrous elements of a net, web, fabric or sheet embedded in a sheet or film (with the particles exposed to enable adsorption or absorption of moisture in contact with the burn), a packet of material with the particles or beads free-flowing within the confines of the packet. The terms particles and beads are not intended to denote any substantive difference in size, shape, or performance of materials and are not asserted as having any distinct differences within the practice of embodiments of the present invention, but are merely alternative terms. The use of only one term does not intend the other term is not equally applicable in the context in which the one term is used. The porous particles and porous beads may also be provided as part of a patch system, with a fibrous network associated with the particles to provide a high level of structural integrity and strength to the applied assembly over the burn, even before clotting has occurred.

The porous particles may easily be associated with or carry additional, but optional, burn treating materials or ingredients. For example, it would be desirable to provide the porous particles with antibiotics, antifungal agents (especially where application may be in a tropical environment), topical pain reducing medication, pharmaceuticals, anti-inflammatants, tissue enzyme inhibitors (e.g., epsilon aminocaproic acid, to reduce tissue enzyme production which weakens the clot), and the like. Existing materials which promote clotting would be helpful, such as thrombin, fibrinogen, aprotinin, fibronectin, and factor XIII. However, one of the advantages of the materials which may be used (excluding those derived from animals) is they are not made from animal components as are the typical clotting or burn treatment materials noted above. As there is always a potential for animal based materials being a source of infection themselves (e.g., viral infection, spongiform encephalopathy, allergic reactions, etc.), the avoidance of animal based products, which can be easily accomplished in the practice of embodiments of the present invention, is desirable.

Polysaccharide components for the porous particles and porous beads of embodiments of the present invention may often be made from cross-linked polysaccharides, such as cross-linked dextran (polybeta-1,6-anhydroglucose). Dextran is a high molecular weight, water-soluble polysaccharide. It is not metabolized by humans, is non-toxic, and is well tolerated by tissue in most animals, including most humans. There have even been extensive use of solubilized dextrans as plasma substitutes. The Sephadex® beads specifically mentioned in the description of particularly useful polysaccharides comprise dextran crosslinked with epichlorihydrin. These beads are available in a variety of bead sizes (e.g., 10 to 100 micrometers, with a range of pore size). It is believed pore sizes on the order of from 5 to 75% of volume may be commercially available and can be expanded to from 5 to 85% by volume or manufactured with those properties from amongst the type of beads described above. The sizes of the pores may also be controlled to act as molecular sieves, the pore size being from 0.5% or 1 to 15% of the largest diameter of the particles or beads. The Sephaex® beads are promoted as having controlled pore sizes for molecular weight cutoff of molecules during use as a sieve, e.g., with cutoff being provided at different intervals between about 5,000 Daltons and 200,000 Daltons. For example, there are cutoff values specifically for molecular weight sizes of greater than 75,000 Daltons. This implies a particle size of specifically about 10 to 40 microns. These beads will rapidly absorb moisture, swelling to several times their original diameter and volume (e.g., from 1.2 to as much as five times their volume).

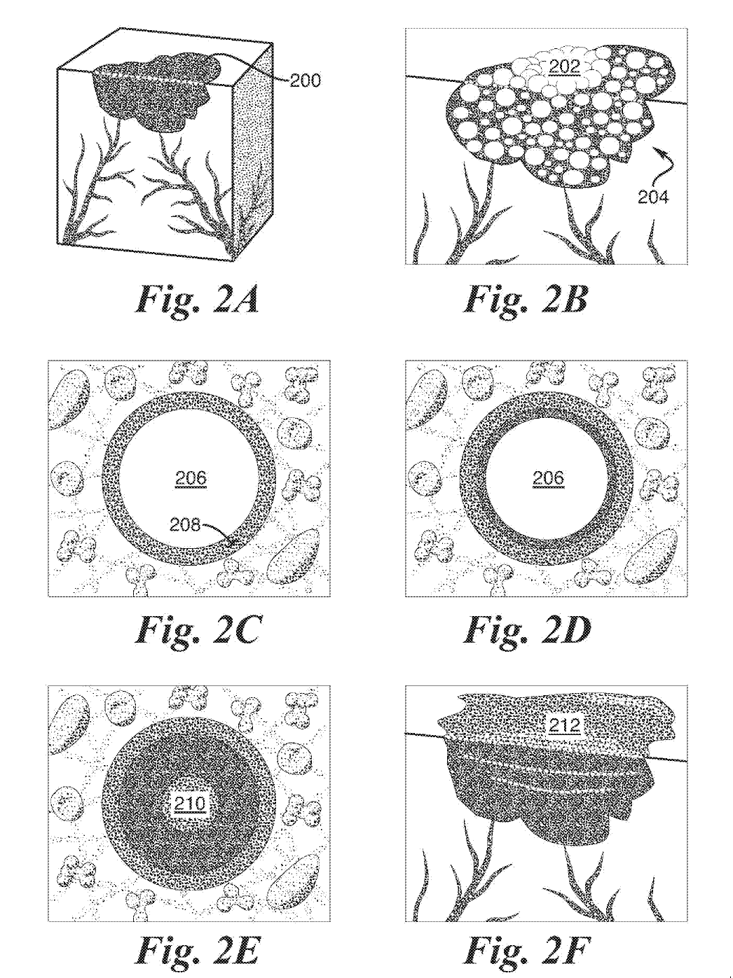

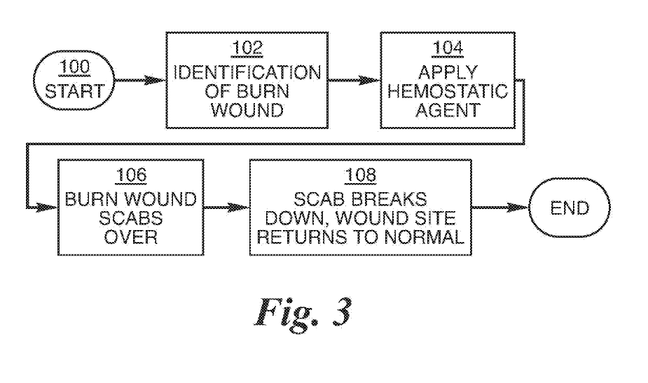

With reference to FIGS. 2A-F and 3, diagrammatic representations of a burn wound being treated by a topical hemostatic agent in an embodiment of the present invention is shown. Burn wound treatment process 100 can be initiated by recognition of a burn wound 200 by a medical responder or even the burn victim at state 102. At state 104, a medical responder or even the victim can begin to apply microporous molecular sieve particles 202 of hemostatic agent 204 to wound 200. Microporous molecular sieve particles 202 can be in a powder form and can be applied to wound site 200 even in the presence of heavy pusing or bleeding.

Application of molecular sieve agent 204 can be in the form of a spray or the powder can be contained and applied with a variety of tools including syringes, endo-surgical applicators, nasal surgical applicators, and reabsorbable dressings. As shown in FIG. 2C, the molecular exclusion properties of bead 206 create a high concentration of proteins and other constituents 208 on the exterior surface as the moisture is absorbed. Bead 206, acting as a molecular sieve, absorbs fluids as proteins and cellular constituents adhere to its surface. Beads 206 accelerate the normal clotting and scabbing cascade. Expanded beads 210 and their load of proteins and solids become part of a clot or scab (state 106) the inventor has found to be more resilient than a purely natural scab. As scab 212 breaks down, beads 210 breakdown into sugars and are absorbed enzymatically, leaving no trace of material at wound 200 within a very short period of time at state 108.

Thus, embodiments of the TOPICAL BURN TREATMENT are disclosed. One skilled in the art will appreciate the present teachings can be practiced with embodiments other than those disclosed. The disclosed embodiments are presented for purposes of illustration and not limitation, and the present teachings are limited only by the claims follow.

What is claimed is:

1. A method for treating a burn, comprising the steps of:
identifying a burn wound; and
applying a molecular sieve agent to the burn wound.

2. The method of claim 1, wherein the molecular sieve agent is Microporous Polysaccharide Hemispheres.

3. The method of claim 2, wherein the Microporous Polysaccharide Hemispheres are located on an interior of a treatment bandage to be placed on the burn wound.

4. The method of claim 2, wherein the Microporous Polysaccharide Hemispheres draw moisture from the burn wound to create a scab over the burn wound.

5. The method of claim 4, wherein the Microporous Polysaccharide Hemispheres and the scab begin to break down over time and the burn wound returns to a normal skin condition.

6. A method for reducing pain associated with a burn, comprising the steps of:
identifying a burn wound; and
applying a molecular sieve agent to the burn wound.

7. The method of claim 6, wherein the molecular sieve agent is combined with an antibacterial agent.

8. The method of claim 6, wherein the molecular sieve agent is Microporous Polysaccharide Hemispheres.

9. The method of claim 6, wherein the molecular sieve agent is applied in powdered form and spread over the burn wound and allowed to set.

10. The method of claim 6, wherein the molecular sieve agent can be applied to the burn wound as a free flowing powder of the particles, a dry spray of particles, a moist spray or aerosol of the particles, as an association of particles in or on a carrier.

11. A method of reducing the possibility of infection in a burn, comprising the steps of:
identifying a burn wound; and
applying topically a molecular sieve agent to the burn wound.

12. The method of claim 11, wherein the molecular sieve agent is Microporous Polysaccharide Hemispheres.

13. The method of claim 12, further comprising the step of allowing the burn wound to scab over due to absorption of moisture.

14. The method of claim 12, wherein the molecular sieve agent is in combined with a topical pain reliever.

15. The method of claim 12, wherein the burn wound can be a first, second, or third degree burn wound.

16. A method of treating burns, comprising the steps of:
identifying a burn wound; and
applying topically a molecular sieve agent to the burn wound.

17. The method of claim 16, wherein the molecular sieve agent has porous part dimensions of from about 0.5 to 1000 micrometers to at least a portion of said burn wound, allowing said porous particles to remain in contact with said burn wound in said wound while clotting initiates in said burn wound.

18. The method of claim 17, wherein said porous particles have molecular sieve cutoff values between about 5,000 Daltons and 200,000 Daltons.

19. The method of claim 18, wherein said pores comprise from 5 to 75% b of the volume of the porous particles.

20. The method of claim 18, wherein the pores comprise from 5 to 35% of the volume of the porous particles.
